(19) United States
(12) Patent Application Publication
(10) Pub. No.: US 2007/0292855 A1
(43) Pub. Date: Dec. 20, 2007

(54) Method and CMOS-based device to analyze molecules and nanomaterials based on the electrical readout of specific binding events on functionalized electrodes

(75) Inventors: Valery M. Dubin, Portland, OR (US); Florian Gstrein, Portland, OR (US); Jonathan Lueker, Portland, OR (US)

Correspondence Address: VALERY M. DUBIN, 5388 NW LIANNA WAY, PORTLAND, OR, 97007, US

(73) Assignee: Intel Corporation, Santa Clara, CA (US)

(21) Appl. No.: 11/207,000

(22) Filed: Aug. 19, 2005

(51) Int. Cl. C12Q 1/68; C12M 3/00

(52) U.S. Cl. 435/6; 435/287.2; 205/777

(57) ABSTRACT

A device having a functionalized electrode having a probe molecule, wherein the device has an ability to electrically detect a molecular binding event between the probe molecule and a target molecule by a polarization change of the functionalized electrode is disclosed. The device could also include an unfunctionalized electrode that does not have the probe molecule and the device could have an ability to electrically detect the molecular binding event between the probe molecule and the target molecule by a polarization change between the functionalized electrode and the unfuctionalized electrode.

### RELATED APPLICATIONS

This application is related to U.S. application Ser. No. 11/144,679, filed Jun. 6, 2005.

### FIELD OF INVENTION

The embodiments of the invention relate to a CMOS-based device to analyze molecules and nanomaterials based on the electrical readout of specific binding events on a functionalized electrode, and it relates to methods and apparatus for preparing such CMOS-based devices. The invention transcends several scientific disciplines such as polymer chemistry, biochemistry, molecular biology, medicine and medical diagnostics.

### BACKGROUND

Molecular recognition (also called a binding event) is fundamental to every cellular event: transcription, translation, signal transduction, viral and bacterial infection and immune response are all mediated by selective recognition events. Thus, developing a better understanding of detecting the binding events of molecules is of significant importance. In the embodiments of this invention, the binding events are detected on a microarray chip having functionalized and unfunctionalized electrodes.

Synthesis of a functionalized electrode having polymer arrays on an electrode of a microarray chip is known. Examples of such polymer arrays include nucleic acid arrays, peptide arrays, and carbohydrate arrays.

One method of preparing functionalized electrodes of polymer arrays on microarray chips involves photolithographic techniques using photocleavable protecting groups. Briefly, the method includes attaching photoreactive groups to the surface of a substrate, exposing selected regions of the substrate to light to activate those regions, attaching a monomer with a photo removable group to the activated regions, and repeating the steps of activation and attachment until macro molecules of the desired length and sequence are synthesized.

Additional methods and techniques applicable to prepare a functionalized electrode include electrochemical synthesis. One example includes providing a porous substrate with an electrode therein, placing a molecule having a protected chemical group in proximity of the porous substrate, placing a buffering solution in contact with the electrode and the porous substrate to prevent electrochemically generated reagents from leaving the locality of the electrode (the use of confinement electrodes to prevent reagents from diffusing away have also been described), applying a potential to the electrode to generate electrochemical reagents capable of deprotecting the protected chemical functional group of the molecule, attaching the deprotected chemical functional group to the porous substrate or a molecule on the substrate, and repeating the above steps until polymers of the desired length and sequence are synthesized.

The molecular recognition events typically are detected through optical readout of fluorescent labels attached to a target molecule that is specifically attached or hybridized to a probe molecule. These molecular recognition event methods are difficult to implement and miniaturize because they rely on the use of optical labels and require large or expensive instrumentation.

### BRIEF DESCRIPTION OF THE DRAWINGS

FIG. 1 shows a schematic diagram of an embodiment of a device of this invention for detection of polarization changes to monitor binding events.

FIG. 2 shows a circuit diagram of a device used to study polarization changes between an active portion of the device (DNA/probe modified electrode) and a reference portion of the device (no probe present). All components are standard solid state devices: differential amplifier, 100 fF MOS capacitors (high pass filter to discard leakage), and CMOS switches.

FIG. 3 shows probe molecules immobilized on the surface of the electrode. When target molecules, such as a complimentary DNA ployanions, are introduced to the solution, the target strand will bond to the DNA probe molecule on the surface via well know Watson/Crick base pair interactions. This means that more anions adsorb to the surface in excess of the equilibrium surface coverage of DNA. Thus, the charge density in the solution side will become nonzero. Since the electrode is more polarizable than the solution a countercharge will be induced in the electrode and this charge can be measured. Note that the circuit is closed through a counter electrode.

FIG. 4 shows schematic of floating electrodes before and after hybridization.

FIG. 5 shows an embodiment of the test device to study temperature-induced de-hybridization events.

FIG. 6(a) shows a via electrode and a surface electrode and (b) shows the generated charge calculated for single-stranded DNA with 25 base pairs undergoing hybridization with a complimentary strand. The amount of the generated charge is 50×1.6×10⁻¹⁹ Coulombs.

FIG. 7 shows a device to trap single strand DNA (ssDNA) into a channel in the electrodes.

FIG. 8 shows a process of manufacturing the device to trap ssDNA.

### DETAILED DESCRIPTION

Nucleic acids (DNA and RNA) can form double-stranded molecules by hybridization, that is, complementary base pairing. The specificity of nucleic acid hybridization is such that the detection of molecular and/or nanomaterials binding events can be done through electrical readout of polarization changes caused by the interaction of charged target molecules (DNA, RNA, proteins, for example.) and chemically modified nanomaterials (carbon nanotubes, nanowires, nanoparticles functionalized with DNA, for example) with complementary molecular probes (DNA, RNA, anti-body, for example) attached to the electrodes (such as Au, Pt, for example). This specificity of complementary base pairing also allows thousands of hybridization to be carried out simultaneously in the same experiment on a DNA chip (also called a DNA array).

Polarization change (for example induced by negatively charged DNA) can be further amplified by the use of enzyme labeled target molecules. Molecular probes are immobilized on the surface of individually addressable electrode arrays through the surface functionalization techniques. Electrodes allow polarization changes to be electrically detected.

The polymer arrays of the embodiment of the invention could be a DNA array (collections of DNA probes on a shared base) comprising a dense grid of spots (often called elements or pads) arranged on a miniature support. Each spot could represent a different gene.

The probe in a DNA chip is usually hybridized with a complex RNA or cDNA target generated by making DNA copies of a complex mixture of RNA molecules derived from a particular cell type (source). The composition of such a target reflects the level of individual RNA molecules in the source. The intensities of the signals resulting from the binding events from the DNA spots of the DNA chip after hybridization between the probe and the target represent the relative expression levels of the genes of the source.

The DNA chip could be used for differential gene expression between samples (e.g., healthy tissue versus diseased tissue) to search for various specific genes (e.g., connected with an infectious agent) or in gene polymorphism and expression analysis. Particularly, the DNA chip could be used to investigate expression of various genes connected with various diseases in order to find causes of these diseases and to enable accurate treatments.

Using an embodiment of the polymer array of the invention, one could find a specific segment of a nucleic acid of a gene, i.e., find a site with a particular order of bases in the examined gene. This detection could be performed by using a diagnostic polynucleotide made up of short synthetically assembled single-chained complementary polynucleotide—a chain of bases organized in a mirror order to which the specific segment of the nucleic acid would attach (hybridize) via A-T or G-C bonds.

The practice of the embodiments of the invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, detection of hybridization using a label. Specific illustrations of suitable techniques can be had by reference to the example herein below. However, other equivalent conventional procedures can, of course, also be used.

As used in the specification and claims, the singular forms “a”, “an” and “the” include plural references unless the context clearly dictates otherwise. For example, the term “an array” may include a plurality of arrays unless the context clearly dictates otherwise.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray would generally contain spot sizes of less than 300 microns.

“Solid support,” “support,” and “substrate” refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain aspects, the solid support(s) will take the form of beads, resins, gels, microspheres, or other geometric configurations.

The term “probe” or “probe molecule” refers to a molecule attached to the substrate of the array, which is typically cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) The polynucleotide probes require only the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism.

The term “target” or “target molecule” refers to a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires and nanoparticles. The target molecule may be fluorescently labeled DNA or RNA.

The terms “die,” “polymer array chip,” “DNA array,” “array chip,” “DNA array chip,” “bio-chip” or “chip” are used interchangeably and refer to a collection of a large number of probes arranged on a shared substrate which could be a portion of a silicon wafer, a nylon strip or a glass slide.

The term “molecule” generally refers to a macromolecule or polymer as described herein. However, arrays comprising single molecules, as opposed to macro molecules or polymers, are also within the scope of the embodiments of the invention.

“Predefined region” or “spot” or “pad” refers to a localized area on a solid support which is, was, or is intended to be used for formation of a selected molecule and is otherwise referred to herein in the alternative as a “selected” region. The predefined region may have any convenient shape, e.g., circular, rectangular, elliptical, wedge-shaped, etc. For the sake of brevity herein, “predefined regions” are sometimes referred to simply as “regions” or “spots.” In some embodiments, a predefined region and, therefore, the area upon which each distinct molecule is synthesized is smaller than about 1 cm² or less than 1 mm², and still more preferably less than 0.5 mm². In most preferred embodiments the regions have an area less than about 10,000 μm² or, more preferably, less than 100 μm². Additionally, multiple copies of the polymer will typically be synthesized within any preselected region. The number of copies can be in the thousands to the millions. More preferably, a die of a wafer contains at least 400 spots in, for example, an at least 20×20 matrix. Even more preferably, the die contains at least 2048 spots in, for example, an at least 64×32 matrix, and still more preferably, the die contains at least 204,800 spots in, for example, an at least 640×320 array. A spot could contain an electrode to generate an electrochemical reagent, a working electrode to synthesize a polymer and a confinement electrode to confine the generated electrochemical reagent. The electrode to generate the electrochemical reagent could be of any shape, including, for example, circular, flat disk shaped and hemisphere shaped.

An “electrode” is a body or a location at which an electrochemical reaction occurs. The term “electrochemical” refers to an interaction or interconversion of electric and chemical phenomena.

A “functionalized electrode” is an electrode of a microchip array having a probe molecule that has a specific chemical affinity to a target molecule. An “unfunctionalized electrode” is an electrode of a microchip array having no probe molecule or having a probe molecule that has no specific chemical affinity to a target molecule.

The electrodes used in embodiments of the invention may be composed of, but are not limited to, metals such as iridium and/or platinum, and other metals, such as, palladium, gold, silver, copper, mercury, nickel, zinc, titanium, tungsten, aluminum, as well as alloys of various metals, and other conducting materials, such as, carbon, including glassy carbon, reticulated vitreous carbon, basal plane graphite, edge plane graphite and graphite. Doped oxides such as indium tin oxide, and semiconductors such as silicon oxide and gallium arsenide are also contemplated. Additionally, the electrodes may be composed of conducting polymers, metal doped polymers, conducting ceramics and conducting clays. Among the metals, platinum and palladium are especially preferred because of the advantageous properties associated with their ability to absorb hydrogen, i.e., their ability to be “preloaded” with hydrogen before being used in the methods of the invention.

The electrodes may be connected to an electric source in any known manner. Preferred ways of connecting the electrodes to the electric source include CMOS (complementary metal oxide semiconductor) switching circuitry, radio and microwave frequency addressable switches, light addressable switches, direct connection from an electrode to a bond pad on the perimeter of a semiconductor chip, and combinations thereof. CMOS switching circuitry involves the connection of each of the electrodes to a CMOS transistor switch. The switch could be accessed by sending an electronic address signal down a common bus to SRAM (static random access memory) circuitry associated with each electrode. When the switch is “on”, the electrode is connected to an electric source. Radio and microwave frequency addressable switches involve the electrodes being switched by a RF or microwave signal. This allows the switches to be thrown both with and/or without using switching logic. The switches can be tuned to receive a particular frequency or modulation frequency and switch without switching logic. Light addressable switches are switched by light. In this method, the electrodes can also be switched with and without switching logic. The light signal can be spatially localized to afford switching without switching logic. This could be accomplished, for example, by scanning a laser beam over the electrode array; the electrode being switched each time the laser illuminates it.

In some aspects, a predefined region can be achieved by physically separating the regions (i.e., beads, resins, gels, etc.) into wells, trays, etc.

A “protecting group” is a moiety which is bound to a molecule and designed to block one reactive site in a molecule, but may be spatially removed upon selective exposure to an activator or a deprotecting reagent. Several examples of protecting groups are known in the literature. The proper selection of protecting group (also known as protective group) for a particular synthesis would be governed by the overall methods employed in the synthesis. Activators include, for example, electromagnetic radiation. ion beams, electric fields, magnetic fields, electron beams, x-ray, and the like. A deprotecting reagent could include, for example, an acid, a base or a free radical. Protective groups are materials that bind to a monomer, a linker molecule or a pre-formed molecule to protect a reactive functionality on the monomer, linker molecule or pre-formed molecule, which may be removed upon selective exposure to an activator, such as an electrochemically generated reagent. Protective groups that may be used in accordance with an embodiment of the invention preferably include all acid and base labile protecting groups. For example, peptide amine groups are preferably protected by t-butyloxycarbonyl (BOC) or benzyloxycarbonyl (CBZ), both of which are acid labile, or by 9-fluorenylmethoxycarbonyl (FMOC), which is base labile. Additionally, hydroxyl groups on phosphoramidites may be protected by dimethoxytrityl (DMT), which is acid labile. Exocyclic amine groups on nucleosides, in particular on phosphoramidites, are preferably protected by dimethylformamidine on the adenosine and guanosine bases, and isobutyryl on the cytidine bases, both of which are base labile protecting groups. This protection strategy is known as fast oligonucleotide deprotection (FOD).

Any unreacted deprotected chemical functional groups may be capped at any point during a synthesis reaction to avoid or to prevent further bonding at such molecule. Capping groups “cap” deprotected functional groups by, for example, binding with the unreacted amino functions to form amides. Capping agents suitable for use in an embodiment of the invention include: acetic anhydride, n-acetylimidizole, isopropenyl formate, fluorescamine, 3-nitrophthalic anhydride and 3-sulfoproponic anhydride. Of these, acetic anhydride and n-acetylimidizole are preferred.

Additional protecting groups that may be used in accordance with an embodiment of the invention include acid labile groups for protecting amino moieties: tertbutyloxycarbonyl, tert-amyloxycarbonyl, adamantyloxycarbonyl, 1-methylcyclobutyloxycarbonyl, 2-(p-biphenyl)propyl(2)oxycarbonyl, 2-(p-phenylazophenylyl)propyl(2)oxycarbonyl, alpha.,.alpha.-dimethyl-3,5-dimethyloxybenzyloxy-carbonyl, 2-phenylpropyl(2)oxycarbonyl, 4-methyloxybenzyloxycarbonyl, benzyloxycarbonyl, furfuryloxycarbonyl, triphenylmethyl (trityl), p-toluenesulfenylaminocarbonyl, dimethylphosphinothioyl, diphenylphosphinothioyl, 2-benzoyl-1-methylvinyl, o-nitrophenylsulfenyl, and 1-naphthylidene; as base labile groups for protecting amino moieties: 9-fluorenylmethyloxycarbonyl, methylsulfonylethyloxycarbonyl, and 5-benzisoazolylmethyleneoxycarbonyl; as groups for protecting amino moieties that are labile when reduced: dithiasuccinoyl, p-toluene sulfonyl, and piperidino-oxycarbonyl; as groups for protecting amino moieties that are labile when oxidized: (ethylthio)carbonyl; as groups for protecting amino moieties that are labile to miscellaneous reagents, the appropriate agent is listed in parenthesis after the group: phthaloyl(hydrazine), trifluoroacetyl(piperidine), and chloroacetyl(2-aminothiophenol); acid labile groups for protecting carboxylic acids: tert-butyl ester; acid labile groups for protecting hydroxyl groups: dimethyltrityl; and basic labile groups for protecting phosphotriester groups: cyanoethyl.

An “electrochemical reagent” refers to a chemical generated at a selected electrode by applying a sufficient electrical potential to the selected electrode and is capable of electrochemically removing a protecting group from a chemical functional group. The chemical group would generally be attached to a molecule. Removal of a protecting group, or “deprotection,” in accordance with the invention, preferably occurs at a particular portion of a molecule when a chemical reagent generated by the electrode acts to deprotect or remove, for example, an acid or base labile protecting group from the molecule. This electrochemical deprotection reaction may be direct, or may involve one or more intermediate chemical reactions that are ultimately driven or controlled by the imposition of sufficient electrical potential at a selected electrode.

Electrochemical reagents that can be generated electrochemically at an electrode fall into two broad classes: oxidants and reductants. Oxidants that can be generated electrochemically, for example, include iodine, iodate, periodic acid, hydrogen peroxide, hypochlorite, metavanadate, bromate, dichromate, cerium (IV), and permanganate ions. Reductants that can be generated electrochemically, for example, include chromium (II), ferrocyanide, thiols, thiosulfate, titanium (III), arsenic (III) and iron (II) ions. The miscellaneous reagents include bromine, chloride, protons and hydroxyl ions. Among the foregoing reagents, protons, hydroxyl, iodine, bromine, chlorine and thiol ions are preferred.

The generation of and electrochemical reagent of a desired type of chemical species requires that the electric potential of the electrode that generates the electrochemical reagent have a certain value, which may be achieved by specifying either the voltage or the current. There are two ways to achieve the desired potential at this electrode: either the voltage may be specified at a desired value or the current can be determined such that it is sufficient to provide the desired voltage. The range between the minimum and maximum potential values could be determined by the type of electrochemical reagent chosen to be generated.

An “activating group” refers to those groups which, when attached to a particular chemical functional group or reactive site, render that site more reactive toward covalent bond formation with a second chemical functional group or reactive site.

A “polymeric brush” ordinarily refers to polymer films comprising chains of polymers that are attached to the surface of a substrate. The polymeric brush could be a functionalized polymer films which comprise functional groups such as hydroxyl, amino, carboxyl, thiol, amide, cyanate, thiocyanate, isocyanate and isothio cyanate groups, or a combination thereof, on the polymer chains at one or more predefined regions. The polymeric brushes of the embodiment of the invention are capable of attachment or stepwise synthesis of macro molecules thereon.

A “linker” molecule refers to any of those molecules described supra and preferably should be about 4 to about 40 atoms long to provide sufficient exposure. The linker molecules may be, for example, aryl acetylene, ethylene glycol oligomers containing 2-10 monomer units, diamines, diacids, amino acids, among others, and combinations thereof. Alternatively, the linkers may be the same molecule type as that being synthesized (i.e., nascent polymers), such as polynucleotides, oligopeptides, or oligosaccharides.

The linker molecule or substrate itself and monomers used herein are provided with a functional group to which is bound a protective group. Generally, the protective group is on the distal or terminal end of a molecule. Preferably, the protective group is on the distal or terminal end of the linker molecule opposite the substrate. The protective group may be either a negative protective group (i.e., the protective group renders the linker molecules less reactive with a monomer upon exposure) or a positive protective group (i.e., the protective group renders the linker molecules more reactive with a monomer upon exposure). In the case of negative protective groups, there could be an additional step of reactivation. In some embodiments, this will be done by heating.

The polymer brush or the linker molecule may be provided with a cleavable group at an intermediate position, which group can be cleaved with an electrochemically generated reagent. This group is preferably cleaved with a reagent different from the reagent(s) used to remove the protective groups. This enables removal of the various synthesized polymers or nucleic acid sequences following completion of the synthesis. The cleavable group could be acetic anhydride, n-acetylimidizole, isopropenyl formate, fluorescamine, 3-nitrophthalic anhydride and 3-sulfoproponic anhydride. Of these, acetic anhydride and n-acetylimidizole are preferred.

The polymer brush or the linker molecule could be of sufficient length to permit polymers on a completed substrate to interact freely with binding entities (monomers, for example) exposed to the substrate. The polymer brush or the linker molecule, when used, could preferably be long enough to provide sufficient exposure of the functional groups to the binding entity. The linker molecules may include, for example, aryl acetylene, ethylene glycol oligomers containing from 2 to 20 monomer units, diamines, diacids, amino acids, and combinations thereof. Other linker molecules may be used in accordance with the different embodiments of the present invention and will be recognized by those skilled in the art in light of this disclosure. In one embodiment, derivatives of the acid labile 4,4′-dimethyoxytrityl molecules with an exocyclic active ester can be used in accordance with an embodiment of the invention. More preferably, N-succinimidyl4[bis-(4-methoxyphenyl)-chloromethyl]-benzoate is used as a cleavable linker molecule during DNA synthesis. Alternatively, other manners of cleaving can be used over the entire array at the same time, such as chemical reagents, light or heat.

A “free radical initiator” or “initiator” is a compound that can provide a free radical under certain conditions such as heat, light, or other electromagnetic radiation, which free radical can be transferred from one monomer to another and thus propagate a chain of reactions through which a polymer may be formed. Several free radical initiators are known in the art, such as azo, nitroxide, and peroxide types, or those comprising multi-component systems.

“Living free radical polymerization” is defined as a living polymerization process wherein chain initiation and chain propagation occur without significant chain termination reactions. Each initiator molecule produces a growing monomer chain which continuously propagates until all the available monomer has been reacted. Living free radical polymerization differs from conventional free radical polymerization where chain initiation, chain propagation and chain termination reactions occur simultaneously and polymerization continues until the initiator is consumed. Living free radical polymerization facilitates control of molecular weight and molecular weight distribution. Living free radical polymerization techniques, for example, involve reversible end capping of growing chains during polymerization. One example is atom transfer radical polymerization (ATRP).

A “radical generation site” is a site on an initiator wherein free radicals are produced in response to heat or electromagnetic radiation.

A “polymerization terminator” is a compound that prevents a polymer chain from further polymerization. These compounds may also be known as “terminators,” or “capping agents” or “inhibitors.” Various polymerization terminators are known in the art. In one aspect, a monomer that has no free hydroxyl groups may act as a polymerization terminator.

The term “capable of supporting polymer array synthesis” refers to any body on which polymer array synthesis can-be carried out, e.g., a polymeric brush that is functionalized with functional groups such as hydroxyl, amino, carboxyl etc. These functional groups permit macromolecular synthesis by acting as “attachment points.”

The monomers in a given polymer or macromolecule can be identical to or different from each other. A monomer can be a small or a large molecule, regardless of molecular weight. Furthermore, each of the monomers may be protected members which are modified after synthesis.

“Monomer” as used herein refers to those monomers that are used to a form a polymer. However, the meaning of the monomer will be clear from the context in which it is used. The monomers for forming the polymers of the embodiments of the invention, e.g., a polymeric brush or a linker molecule, have for example the general structure:

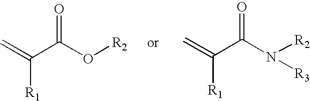

wherein R₁ is hydrogen or lower alkyl; R₂ and R₃ are independently hydrogen, or —Y—Z, wherein Y is lower alkyl, and Z is hydroxyl, amino, or C(O)—R, where R is hydrogen, lower alkoxy or aryloxy.

The term “alkyl” refers to those groups such as methyl, ethyl, propyl, butyl etc, which may be linear, branched or cyclic.

The term “alkoxy” refers to groups such as methoxy, ethoxy, propoxy, butoxy, etc., which may be linear, branched or cyclic.

The term “lower” as used in the context of lower alkyl or lower alkoxy refers to groups having one to six carbons.

The term “aryl” refers to an aromatic hydrocarbon ring to which is attached an alkyl group. The term “aryloxy” refers to an aromatic hydrocarbon ring to which is attached an alkoxy group. One of ordinary skill in the art would readily understand these terms.

Other monomers for preparing macro molecules of the embodiments of the invention are well-known in the art. For example, when the macromolecule is a peptide, the monomers include, but are not restricted to, for example, amino acids such as the L-amino acids, the D-amino acids, the synthetic and/or natural amino acids. When the macromolecule is a nucleic acid, or polynucleotide, the monomers include any nucleotide. When the macromolecule is a polysaccharide, the monomers can be any pentose, hexose, heptose, or their derivatives.

A “monomer addition cycle” is a cycle comprising the chemical reactions necessary to produce covalent attachment of a monomer to a nascent polymer or linker, such as to elongate the polymer with the desired chemical bond (e.g., 5′-3′ phosphodiester bond, peptide bond, etc.). For example, and not to limit the invention, the following steps typically comprise a monomer addition cycle in phosphoramidite-based polynucleotide synthesis: (1) deprotection, comprising removal of the DMT group from a 5′-protected nucleoside (which may be part of a nascent polynucleotide) wherein the 5′-hydroxyl is blocked by covalent attachment of DMT, such deprotection is usually done with a suitable deprotection reagent (e.g., a protic acid: trichloroacetic acid or dichloroacetic acid), and may include physical removal (e.g., washing, such as with acetonitrile) of the removed protecting group (e.g., the cleaved dimethyltrityl group), (2) coupling, comprising reacting a phosphoramidite nucleoside(s), often activated with tetrazole, with the deprotected nucleoside, (3) optionally including capping, to truncate unreacted nucleosides from further participation in subsequent monomer addition cycles, such as by reaction with acetic anhydride and N-methylimidazole to acetylate free 5′-hydroxyl groups, and (4) oxidation, such as by iodine in tetrahydroftiran/water/pyridine, to convert the trivalent phosphite triester linkage to a pentavalent phosphite triester, which in turn can be converted to a phosphodiester via reaction with ammonium hydroxide. Thus, with respect to phosphoramidite synthesis of polynucleotides, the following reagents are typically necessary for a complete monomer addition cycle: trichloroacetic acid or dichloroacetic acid, a phosphoramidite nucleoside, an oxidizing agent, such as iodine (e.g., iodine/water/THF/pyridine), and optionally N-methylimidazole for capping.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or ω-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure.

A “nanomaterial” as used herein refers to a structure, a device or a system having a dimension at the atomic, molecular or macromolecular levels, in the length scale of approximately 1-100 nanometer range. Preferably, a nanomaterial has properties and fuinctions because of the size and can be manipulated and controlled on the atomic level.

A “carbon nanotube” refers to a fuillerene molecule having a cylindrical or toroidal shape. A “fullerene” refers to a form of carbon having a large molecule consisting of an empty cage of sixty or more carbon atoms.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Phosphoramidites protected in this manner are known as FOD phosphoramidites.

Analogs also include protected and/or modified monomers as are conventionally used in polynucleotide synthesis. As one of skill in the art is well aware, polynucleotide synthesis uses a variety of base-protected nucleoside derivatives in which one or more of the nitrogens of the purine and pyrimidine moiety are protected by groups such as dimethoxytrityl, benzyl, tert-butyl, isobutyl and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary polynucleotides (especially RNA) than their unmodified counterparts. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the polynucleotide can also be substituted or modified, for instance with methyl phosphonates, O-methyl phosphates or phosphororthioates. Another example of a polynucleotide comprising such modified linkages for purposes of this disclosure includes “peptide polynucleotides” in which a polyamide backbone is attached to polynucleotide bases, or modified polynucleotide bases. Peptide polynucleotides which comprise a polyamide backbone and the bases found in naturally occurring nucleotides are commercially available.

Nucleotides with modified bases can also be used in the embodiments of the invention. Some examples of base modifications include 2-aminoadenine, 5-methylcytosine, 5-(propyn-1-yl)cytosine, 5-(propyn-1-yl)uracil, 5-bromouracil, 5-bromocytosine, hydroxymethylcytosine, methyluracil, hydroxymethyluracil, and dihydroxypentyluracil which can be incorporated into polynucleotides in order to modify binding affinity for complementary polynucleotides.

Groups can also be linked to various positions on the nucleoside sugar ring or on the purine or pyrimidine rings which may stabilize the duplex by electrostatic interactions with the negatively charged phosphate backbone, or through interactions in the major and minor groves. For example, adenosine and guanosine nucleotides can be substituted at the N² position with an imidazolyl propyl group, increasing duplex stability. Universal base analogues such as 3-nitropyrrole and 5-nitroindole can also be included. A variety of modified polynucleotides suitable for use in the embodiments of the invention are described in the literature.

When the macromolecule of interest is a peptide, the amino acids can be any amino acids, including α, β, or ω-amino acids. When the amino acids are a-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it will be recognized by those of skill that the minimum length of a polynucleotide desired for specific hybridization to a target polynucleotide will depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions will vary depending on the application and are selected in accordance with the general binding methods known in the art.

It is appreciated that the ability of two single stranded polynucleotides to hybridize will depend upon factors such as their degree of complementarity as well as the stringency of the hybridization reaction conditions.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, will remain single-stranded. Conversely, at lower stringency, the probability of formation of a mismatched duplex is increased.

The appropriate stringency that will allow selection of a perfectly-matched duplex, compared to a duplex containing one or more mismatches (or that will allow selection of a particular mismatched duplex compared to a duplex with a higher degree of mismatch) is generally determined empirically. Means for adjusting the stringency of a hybridization reaction are well-known to those of skill in the art.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macro molecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention may be useful in investigating the ligand-binding site on the antibody molecule which combines with the epitope of an antigen of interest; determining a sequence that mimics an antigenic epitope may lead to the-development of vaccines of which the immunogen is based on one or more of such sequences or lead to the development of related diagnostic agents or compounds useful in therapeutic treatments such as for auto-immune diseases (e.g., by blocking the binding of the “anti-self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

The term “complementary” refers to the topological compatibility or matching together of interacting surfaces of a ligand molecule and its receptor. Thus, the receptor and its ligand can be described as complementary, and furthermore, the contact surface characteristics are complementary to each other.

A “scribe line” is typically an “inactive” area between the active dies that provide area for separating the die (usually with a saw). Often, metrology and alignment features populate this area.

A “via” refers to a hole etched in the interlayer of a dielectric which is then filled with an electrically conductive material, preferably tungsten, to provide vertical electrical connection between stacked up interconnect metal lines that are capable of conducting electricity.

“Metal lines” within a die are interconnect lines. Metal interconnect lines do not typically cross the scribe line boundary to electrically connect two dies or, as in the some embodiments of this invention, a multitude of die to one or more wafer pads.

The term “oxidation” means losing electron to oxidize. The term “reduction” means gaining electrons to reduce. The term “redox reaction” refers to any chemical reaction which involves oxidation and reduction.

The term “wafer” means a semiconductor substrate. A wafer could be fashioned into various sizes and shapes. It could be used as a substrate for a microchip. The substrate could be overlaid or embedded with circuitry, for example, a pad, via, an interconnect or a scribe line. The circuitry of the wafer could also serve several purpose, for example, as microprocessors, memory storage, and/or communication capabilities. The circuitry can be controlled by the microprocessor on the wafer itself or controlled by a device external to the wafer.

The term “molecular binding event” means the occurrence of contact between a probe molecule and a target molecule. The devices for detecting a molecular binding event according to the embodiments of the present invention are intended for use in a molecular recognition-based assay for the analysis of a sample suspected of containing one or more target molecules or moieties such as specific nucleic acid sequences. The probe molecules of the array are provided for the purpose of binding and detecting specific target molecules, e.g., nucleic acid sequences. The hybridization between the probe and target nucleic acid sequences may occur through the standard Watson-Crick hydrogen-bonding interactions or other known specific binding interactions known in the art.

The term “polarization change” means a change in the amount ofcharge on an electrode produced by the deposition of a target molecule.

The term “differential amplifier” means a device that amplifies the difference between two input signals (−) and (+). This amplifier is also referred to as a differential-input single-ended output amplifier. It is a precision voltage difference amplifier, and could form the central basis of more sophisticated instrumentation amplifier circuits.

The term “field effect transistor” (FET) is a family of transistors that rely on an electric field to control the conductivity of a “channel” in a semiconductor material. FETs, like all transistors, can be thought of as voltage-controlled resistors. Most FETs are made using conventional bulk semiconductor processing techniques, using the single-crystal semiconductor wafer as the active region, or channel.

The term “CMOS” means complementary metal oxide semiconductor.

One embodiment of the invention is directed to a device comprising a functionalized electrode having a probe molecule, wherein the device has an ability to electrically detect a molecular binding event between the probe molecule and a target molecule by a polarization change of the functionalized electrode. The device could further comprise an unfunctionalized electrode that does not have the probe molecule, wherein the device has an ability to electrically detect the molecular binding event between the probe molecule and the target molecule by a polarization change between the functionalized electrode and the unfunctionalized electrode. Preferably, the probe molecule and the target molecule are label-free. Preferably, the target molecule is a single-stranded DNA, a carbon nanotube functionalized with a DNA, or a nanomaterial and the probe molecule is a single-stranded DNA or a nanomaterial.

More preferably, the device is a CMOS-based charge sensor and the device is not a current-voltage redox sensor. The device could be based on CMOS structures and electrode arrays which are individually fuictionalized with a variety of molecular probes having a specific chemical affinity towards a variety of individually matching/interacting target molecules and chemically modified nanomaterials. The device could electrically detect molecular binding events on the electrode arrays, sense and amplify currents generated during polarization changes at the interface. Electrode arrays could interface with logic devices and serve as charge pumps.

The device could further comprise a differential amplifier to amplify a current generated by the polarization change of the functionalized electrode. The device could further comprise a differential amplifier, wherein the differential amplifier is to amplify a current generated by the polarization change between the functionalized electrode and the unfunctionalized electrode.

In one variation, the device could further comprise a substrate comprising a wafer. The device could further comprise a switch and a capacitor, and wherein the polarization change modulates a gate of the field effect transistor.

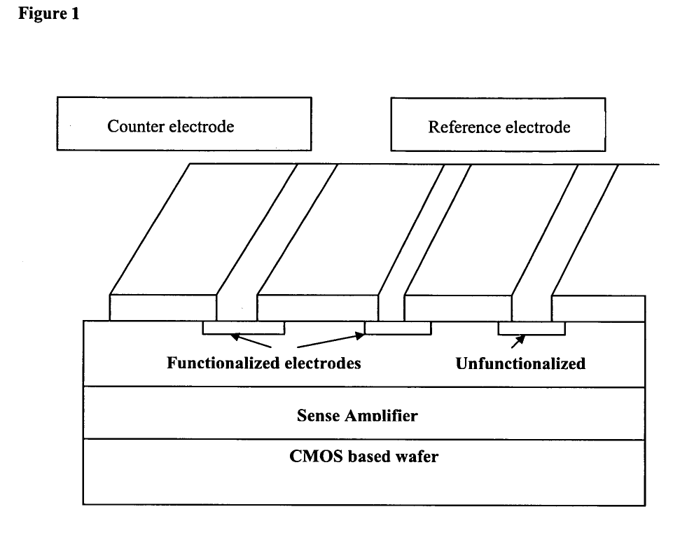

One embodiment of the invention includes the structures of the device built on CMOS-based wafers to electrically detect molecular binding events on an array of electrodes functionalized with probe molecules having specific chemical affinity to target molecules, differential amplifiers to amplify current generated during polarization change on the interface, CMOS switches, MOS capacitors as shown in FIG. 1. The polarization at the electrode/solution interface (for example, electrode material could be Au, Pt) changes due to the binding of charged target molecules (such as but not necessarily limited to single-stranded DNA, carbon nanotubes functionalized with DNA, for example) to specifically adsorbed probe molecules (such as complementary single-stranded DNA immobilized on the electrode surface).

Another embodiment includes a device where the electrodes detect polarization changes which modulate the gate of a field effect transistor. Another embodiment of the invention includes devices that allow polarization changes to be monitored during de-hybridization (reversible recognition) events. In another embodiment of this invention charged biomolecules can be trapped into a channel on an analysis electrode, thereby improving the yield for DNA hybridization detection.

Another embodiment includes the generation of catalytic current on electrodes by coupling enzyme-catalyzed reactions that generate electrical signals, or amplify existing signals, when probe/target recognition events occur. For example, DNA probes can be attached to electrode array devices. These probes can then be overlayed with biotinylated target DNA that will specifically hybridize to the probes. These complexes can then be overlayed with streptavidin-conjugated horseradish peroxidase (HRP) that will specifically bind to the target/probe recognition complex via streptavidin/biotin binding. The resulting complex is well known in the art to catalyze the reduction of H₂O₂, thereby generating catalytic current Such approaches are known in the art as a method for amplifying the binding of conjugated/tagged probe/targets e.g., by several orders of magnitude or shifting the potential of HRP modified electrode of about 0.7V higher (more anodic) than the potential of non-modified electrode.

Another embodiment of the invention is directed at a method of manufacturing a device for electrical detection of the molecular binding events. The method could comprise functionalizing a first electrode with a probe molecule to form a functionalized electrode, not functionalizing a second electrode to form an unfunctionalized electrode, and fabricating a differential amplifier, wherein the device has an ability to electrically detect a molecular binding event between the probe molecule and a target molecule by a polarization change between the functionalized electrode and the unfunctionalized electrode. Preferably, the differential amplifier is to amplify a current generated by the polarization change between the functionalized electrode and the unfunctionalized electrode. The method could further comprise fabricating an interface logic. Preferably, by the method of an embodiment of invention, the device has the ability to electrically detect the molecular binding event without labeling the probe molecule or the target molecule, i.e., even when the probe and target molecules are label-free.

In an embodiment of the invention, the method of fabrication of a device for electrical detection of molecular binding events are: a) fabrication of CMOS structures on the wafer containing amplifiers, interface logic, charge pumps and array of electrodes by using standard fabrication techniques such as lithography, etch, ion implantation, thin films, and packaging, and b) functionalization of electrodes with chemical or bio-chemical probes having specific chemical affinity to target molecules.

Another embodiment of the invention is directed to a method of measuring a molecular binding event between a probe molecule and a target molecule, comprising obtaining a device comprising a functionalized electrode having a probe molecule and an unfunctionalized electrode that does not have the probe molecule, and detecting the molecular binding event between the probe molecule and the target molecule by a polarization change between the functionalized electrode and the unfunctionalized electrode. In a method of measuring the molecular binding event, the device could further comprise a differential amplifier, wherein the differential amplifier is to amplify a current generated by the polarization change between the functionalized electrode and the unfunctionalized electrode. Preferably, the method of measuring the molecular binding event could further comprise modulating a gate of the field effect transistor by the polarization change.

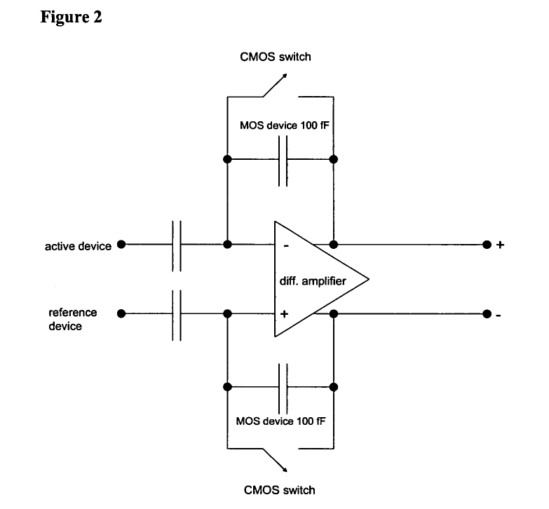

In an embodiment of the invention, the method of electrical detection of molecular binding events are: a) exposure of electrode arrays to target molecules and/or chemically modified. nanomaterials that resulted in specific binding events of target molecules (chemically modified nanomaterials such as for example DNA modified CNT) with molecular probes accompanied by polarization change at the electrode interface; and b) measurement and amplification of currents generated by the polarization change (bridge structures can be used to amplify the electrical signal delta between electrodes undergoing specific binding events vs. electrodes that stay inactive; amplification can be done with sense amplifiers). Amplification can be done with a device depicted in FIG. 2. Another embodiment includes device where the electrodes detect polarization changes which modulate the gate of a field effect transistor.

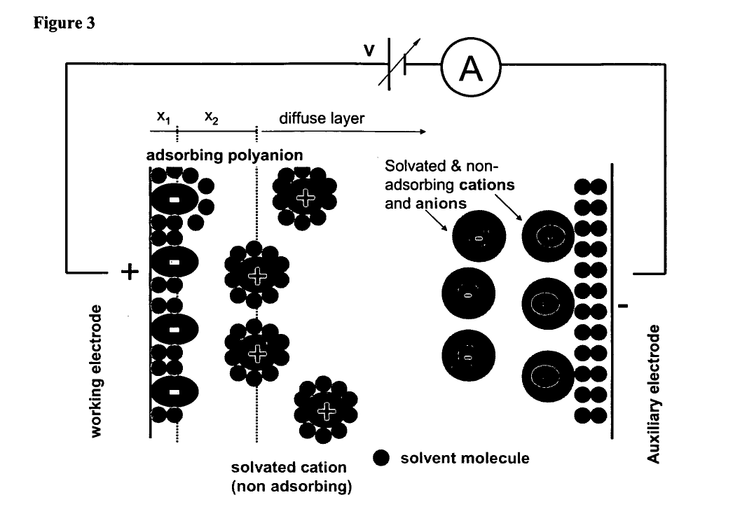

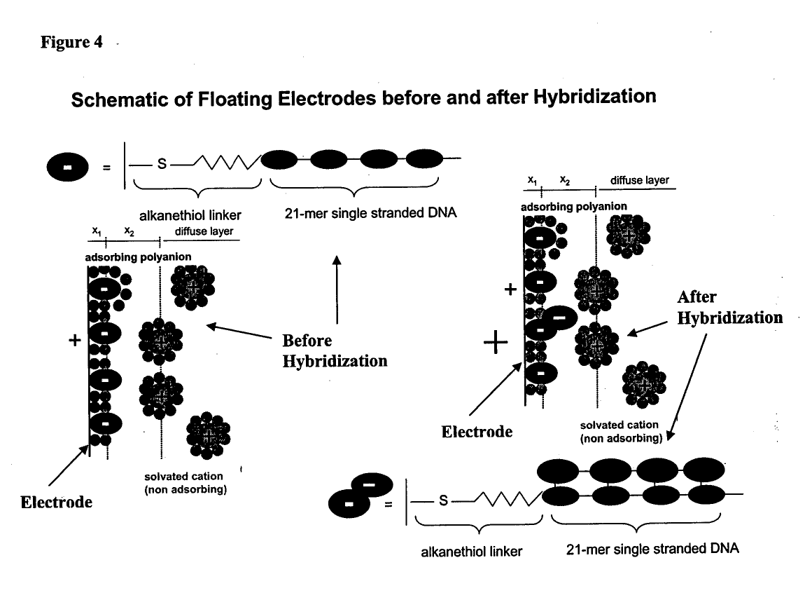

While not being tied to a particular theory, the technical basis for the embodiments of the invention is considered to be as follows. In solution, DNA probe molecules dissociate into DNA polyanions and compensating cations as shown in FIG. 3. These polyanions can be selectively adsorbed to the electrode surface using any known surface modification chemistry such as but not necessarily limited to thiolates, amines, and poly(mercaptopropyl)methylsiloxane. Thus, probe molecules are immobilized on the surface of the electrode. When a target molecule such as a complimentary DNA polyanion is introduced to the solution, the target strand will bond to the DNA probe molecule on the surface via well know Watson/Crick base pair interactions. This means that more anions adsorb to the surface in excess of the equilibrium surface coverage of DNA. The charge density in the solution side will become nonzero. Since the electrode is more polarizable than the solution a countercharge will be induced in the electrode and this charge can be measured (circuit is closed through counter electrode). If the electrode is held at constant surface charge the adsorption of DNA anions is accompanied by a negative potential shift as has been observed in experiments. The current resulting from a polarization change at the interface (dQ/dt) for a single DNA molecule undergoing hybridization is about 10 fA. As shown in FIG. 4, the detection of polarization changes relies on changes to the electrical double layer capacitance at the solution/electrode interface when a charged target molecule binds to a probe molecule.

Preferably, in the embodiments of the invention (with specific reference to FIG. 4), the individual electrode area is about 1 μm²; the electrodes are individually addressable; the interface capacitance between the electrode and solution is about 10 μF/cm²; the voltage change in the double electrical layer of the metal electrode and the layer of charges species in supporting electrolyte solution is about 0.5 V; the total charge on the electrode is about 5×10⁻¹⁴ C (50 fC); the charge change on the interface of the electrode due to binding of DNA is about 10⁻¹⁵ C (1 fC), i.e., change in charge due to binding of DNA is about 0.02×charge stored in the electrical double layer; the binding and debinding event times are of the order of millisecond; a reference electrode can be made that may be covered with a probe molecule which is inactive towards binding with a target molecule; and de-binding can be initiated (enzymatic, temperature induced de-hybridization). The molecular binding event measurements by the embodiment of the invention could be undertaken by measuring the current due to polarization change on the interface (dQ/dt), which is of the order of about 10⁻¹² A (about 1 pA). The measured current could decrease by a factor of 0.01 for each change in the order of magnitude of the electrode size.

Another embodiment of the invention relates to a test device comprising (a) a first metal layer comprising a functionalized electrode comprising a probe polynucleotide and (b) a second metal layer comprising a second electrode that can be resistively heated to cause a target polynucleotide to de-hybridize, wherein the test device is to study de-hybridization of the target polynucleotide. The test device could further comprise a third metal layer comprising a third electrode that can be resistively heated to cause the target polynucleotide to de-hybridize. Preferably, the probe polynucleotide is selected to such that the probe polynucleotide can withstand a temperature of up to about 80° C. Preferably, the test device has an ability to electrically detect a molecular de-binding event between the probe polynucleotide and the target polynucleotide by a polarization change of the functionalized electrode. Preferably, the first metal layer further comprises an unfunctionalized electrode that does not have the probe polynucleotide, wherein the test device has an ability to electrically detect the molecular de-binding event between the probe polynucleotide and the target polynucleotide by a polarization change between the functionalized electrode and the unfunctionalized electrode. More preferably, the test device is to study enzymatic or temperature-induced de-hybridization of the target polynucleotide.

An alternative way of detecting a successful hybridization event is to monitor the polarization changes of double-stranded DNA during de-hybridization (reversible probe/target interactions), since only hybridized DNA can undergo a de-hybridization reaction. The device architecture remains essentially the same. The by far most commonly used method of inducing de-hybridization of double-stranded DNA are so called temperature jump experiments. The temperature is suddenly raised by 20-40° C and de-hybridization is induced. The de-hybridization kinetics of double-stranded DNA following such a temperature jump has been monitored by various techniques such as frequency-resonance energy spectroscopy (FRET), time-resolved fluorescence spectroscopy. Electrical readout of polarization changes resulting from temperature-induced de-hybridization, have the advantage that unlike hybridization, de-hybridization is not a slow and diffusion-controlled process but rather an instantaneous process. Hence charge does not have to be integrated over an extended period of time and thereby be subjected to background noise. Temperature changes can be induced by various techniques such as, constant temperature bath, resistive heating of small volumes containing the electrolyte (the small volume of the liquid ensures rapid heating), laser jump techniques, or by radio-frequency heating of gold nanocrystals covalently attached to double-stranded DNA. One embodiment to induce the de-binding DNA on the surface of electrode uses negative voltage (100 mV+). Integration of the charge change could be done immediately after the debinding event, thus increasing the signal to noise ratio.

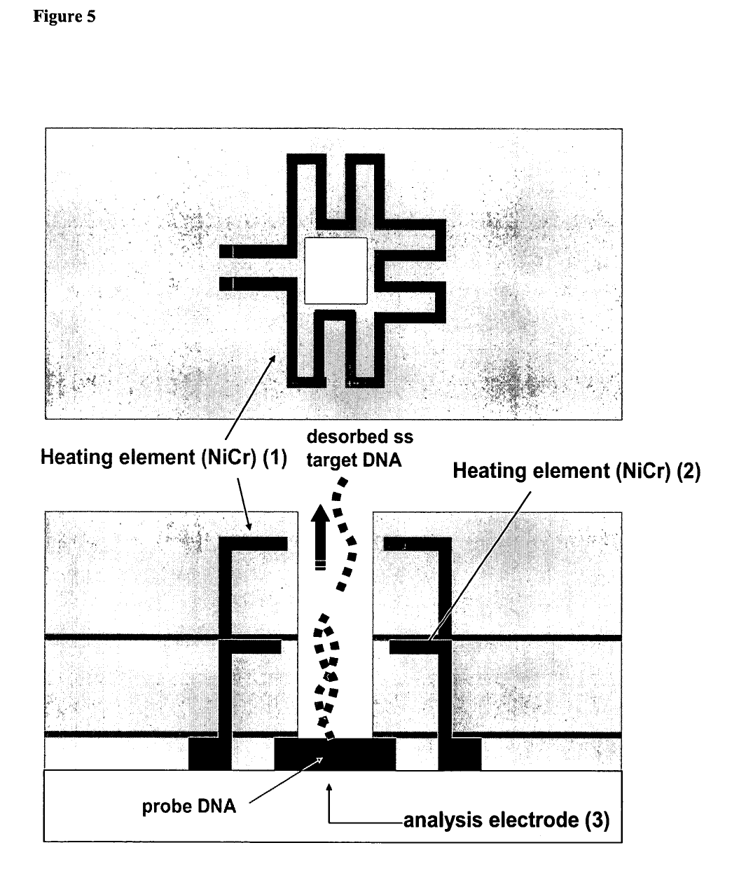

Yet another method for de-binding DNA is resistive heating on a chip. An example of the test device to study temperature-induced de-hybridization events is shown in FIG. 5. This device could have 3 metal layers. The first metal layer contains an analysis electrode (modified with probe DNA and hybridized with target DNA), which is shown as analysis electrode (3) in FIG. 5. The second and third metal layers could contain NiCr electrodes, which are shown as heating electrodes (1) and (2) in FIG. 5, that can be resistively heated causing the target DNA molecule to desorb or de-hybridize. The chemical binding moiety of the probe DNA molecule is chosen such that it can withstand a higher temperature than room temperature. Devices used for signal amplification (see FIG. 2) are not shown in FIG. 5 for clarity.

Another embodiment includes the generation of catalytic current on electrodes by coupling enzyme-catalyzed reactions that generate electrical signals, or amplify existing signals, when probe/target recognition events occur. For example, DNA probes can be attached to electrode array devices. These probes can then be overlayed with biotinylated target DNA that will specifically hybridize to the probes. These complexes can then be overlayed with streptavidin-conjugated horseradish peroxidase (HRP) that will specifically bind to the target/probe recognition complex via streptavidin/biotin binding. The resulting complex is well known in the art to catalyze the reduction of H₂O₂, thereby generating catalytic current (H₂O₂+2H++2e→2H₂O). Such approaches are known in the art as a method for amplifying the binding of conjugated/tagged probe/targets e.g., by several orders of magnitude or shifting the potential of HRP modified electrode of about 0.7V higher (more anodic) than the potential of non-modified electrode.

Another embodiment of the invention relates to a method of manufacturing a circuit comprising attaching a first polynucleotide to a first end of a nanomaterial and hybridizing the first polynucleotide to a second polynucleotide located on a first pad of a die and further comprising attaching a third polynucleotide of a second of the nanomaterial and hybridizing the third polynucleotide to a fourth polynucleotide located on a second pad of the die. Preferably, the nanomaterial forms an electrically conductive path between the first and second pads. More preferably, the nanomaterial is a carbon nanotube. In one variation, the first pad comprises an electrode and the hybridization is coupled with generation of a catalytic current on the electrode.

Yet another embodiment of the invention relates to a die comprising a first pad, a second pad and a nanomaterial connecting the first and second pads, wherein the nanomaterial is connected to the first and second pads by hybridized polynucleotide, wherein the nanomaterial is a carbon nanotube.

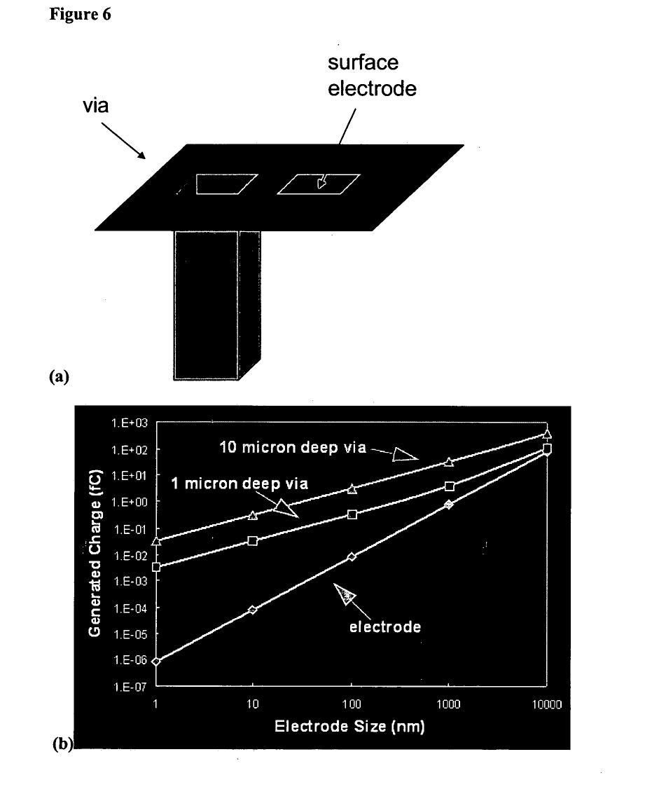

Another embodiment of the invention relates to an electrode having a three dimensional shape of a via having a bottom wall and side walls as shown in FIG. 6(a). Preferably, the via is about 1 to 10,000 micron in width and about 1 to 10 microns in deep. 3D electrodes in which the bottom, walls, and/or tops of the test chamber are manufactured to accommodate the electrical detection technologies described herein are also within the embodiments of the invention. Such electrode structures can be used to increase the surface area available for probe/target recognition and subsequent detection, thereby increasing the sensitivity of these devices. FIG. 6(b) shows the generated charge calculated for single-stranded DNA with 25 base pairs undergoing hybridization with a complimentary strand. The amount of the generated charge is 50×1.6×10⁻¹⁹ Coulombs. The surface coverage of DNA bound to the electrode was estimated conservatively to be on the order of 10¹⁰ DNA molecules per cm² (as high as 10¹² cm⁻² have been reported). A high-aspect ratio via with 10 nm diameter that is 10 microns deep has enough surface area to detect hybridization events of DNA. Surface electrodes should preferably be larger than 1 micron to have a similar read out. A via of 1 micron diameter that is 10 micron deep yields at a minimum 10 fC charge.

Another embodiment of the invention relates to a device for trapping a target molecule comprising a first electrode having a probe molecule or a polymer brush attached to the first electrode, a second electrode and a third electrode, wherein the first, second and third electrodes are independently addressable electrodes, and wherein the second and third electrodes overlap the first electrode and contains a channel above the first electrode to permit the target molecule to be trapped into the channel. Preferably, the target molecule is a charged target molecule and the second and third electrodes have a voltage difference to produce an electric field between the second and third electrodes to ensure that the charged target molecule is attracted into the channel. The probe molecule could be a cDNA probe or a polynucleotide probe. The device could further have a CMOS circuitry comprising a switching scheme for individually addressing the first, second and third electrodes. The device could further include a metal layer between the first electrode and the second electrode and another metal layer between the second electrode and the third electrode. The charged target molecule could be a charged DNA, a charged nanomaterial, or a nanomaterial modified with a charged DNA. In one variation of an embodiment of the invention, one end of the channel that meets the first electrode could be closed and the other end of the channel could be open to ensure movement of a target molecule comprising a DNA through the open end of the channel. Also, in a variation of an embodiment of the device for trapping the target molecule, the second and third electrodes could be ring shaped.

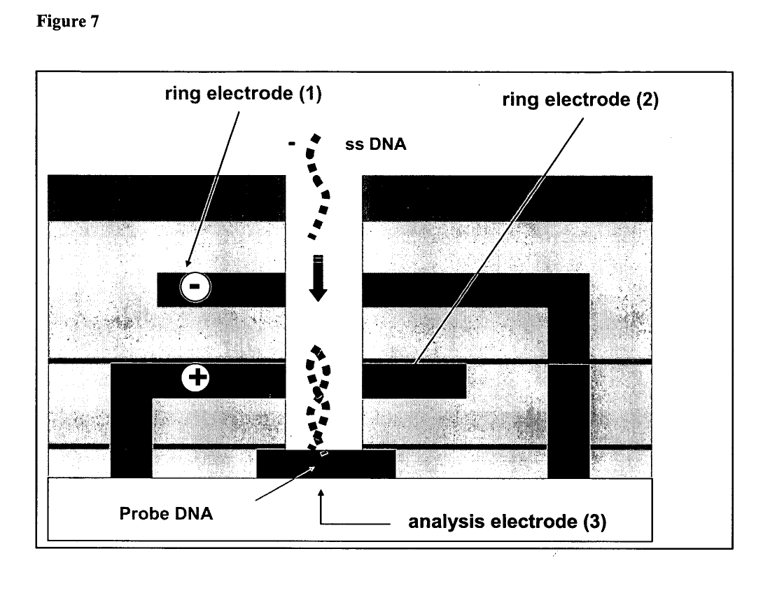

FIG. 7 shows a device to trap single-strand DNA into a channel equipped with an analysis electrode (i.e., the first electrode), which is shown as analysis electrode 3 in FIG. 7. The device could have three independently addressable electrodes on three metal layers. An electric field between ring electrode 2 (positive electrode) and electrode 1 (negative electrode) ensures that negatively charged DNA (or nanomaterials such as CNT, nano-particles Au etc modified with DNA) is forced into the channel containing the probe DNA (immobilized on the surface by appropriate surface chemistry). The device of FIG. 7 utilizes electric fields to trap single-stranded DNA into a channel through the electrodes thereby improving the yield for DNA hybridization detection.

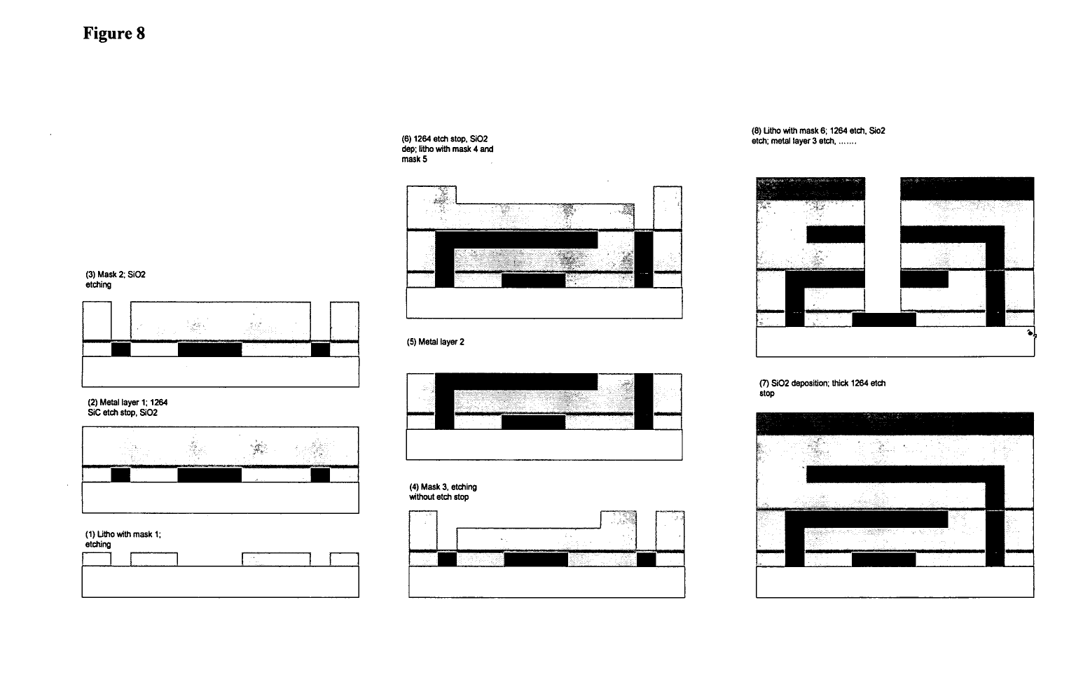

Yet another embodiment of the invention relates to a method for manufacturing a device for trapping a target molecule, comprising forming a first electrode on a substrate, forming a second electrode on the first electrode, a forming third electrode on the second electrode, and forming a channel in the second and third electrodes, wherein the first, second and third electrodes are independently addressable electrodes and the channel above the first electrode is to permit the target molecule to be trapped into the channel. Preferably, the channel ends at the top of the first electrode. The method of manufacturing could further comprise depositing one or more metal layers between any two of the first, second and third electrodes, and depositing one ore more silicon-containing layers. Also, preferably, the second and third electrodes are shaped like a ring. The process flow steps of manufacturing a device for trapping a target molecule are shown in FIG. 8. In step 1, conventional lithographic techniques are used to etch trenches in the interlayer dielectric such as SiO2. These trenches are subsequently filled with, barrier material, seed and metal (Au, Pt, Pd) through standard electroplating techniques—metal layer 1. This is followed by chemical mechanical polishing, etch stop deposition and deposition of an additional layer of interlayer dielectric (step 2). In steps 3-5 the dielectric is patterned with vias and trenches which are subsequently filled with a refractory metal (Au, Pt, Pd) using a dual damascene process. This results in the ring electrode formed by metal layer 2. This is repeated for steps 6-7 leading to the ring electrode formed by metal layer 3. A dielectric layer is deposited in step 2 together with an etch stop layer. The etch stop layer is patterned and a via is etched into the stack of metal and dielectric. This exposes metal layer 1 and the ring electrodes (metal layer 2 and metal layer 3) to the solution.

In a different embodiment of this invention vias and trenches can be filled with copper and subsequently capped with a noble or refractory metal (Au, Pt, Pd) through various electroless plating techniques.

The embodiments of the invention could use silicon technology to fabricate interconnects for silicon chips to enable on-die synthesis of polymers such as DNA, peptides, and DNA-functionalized complementary nucleotide. Optionally, the embodiments of the invention could use wafer processing cluster tools (process instruments) for synthesis. Typically, in volume silicon processing, a manufacturing line has a cluster of instruments (several identical instruments). Each can support a process step or multiple process steps. By the embodiments of the invention, polymer synthesis can be treated as another process step in a device manufacturing line. A cluster of instruments can be configured within a facility to perform wafer level synthesis for efficient high volume manufacturing.

The devices of the embodiments of the invention may be formed by any suitable means of manufacture, including semiconductor manufacturing methods, microforming processes, molding methods, material deposition methods, etc., or any suitable combination of such methods. In certain embodiments one or more of the electrodes and/or the pad may be formed via semiconductor manufacturing methods on a semiconductor substrate. Thin film inorganic coatings may be selectively deposited on portions of the substrate and/or pad surface. Examples of suitable deposition techniques include vacuum sputtering, electron beam deposition, solution deposition, and chemical vapor deposition. The inorganic coatings may perform a variety of functions. For example, the coatings may be used to increase the hydrophilicity of a surface or to improve high temperature properties. Conductive coatings may be used to form electrodes. Coatings may be used to provide a physical barrier on the surface, e.g. to retain fluid at specific sites on the surface. The devices used in the present invention may be fabricated according to procedures well-known in the arts of microarray and semiconductor device manufacturing.

In some embodiments the probes may be selected from biomolecules, such as polypeptides, polynucleotides, glycoproteins, polysaccharides, hormones, growth factors, peptidoglycans, or the like. The probe could be natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. In embodiments employing oligonucleotide probes, the probes may be synthesized, in situ, on the surface of the pad in either the 3′ to 5′ or 5′ to 3′ direction using the 3′-p-cyanoethyl-phosphoramidites or 5′-β-cyanoethyl-phosphoramidites and related chemistries known in the art. In situ synthesis of the oligonucleotides may also be performed in the 5′ to 3′ direction using nucleotide coupling chemistries that utilize 3′-photo removable protecting groups. Alternatively, the oligonucleotide probes may be synthesized on the standard controlled pore glass (CPG) in the 3′ to 5′ direction using 3′-β-cyanoethyl-phosphoramidites and related chemistries and incorporating a primary amine or thiol functional group onto the 5′ terminus of the oligonucleotide. The oligonucleotides may then be covalently attached to the pad surface via their 5′ termini using thiol or amine-dependent coupling chemistries known in the art. The density of the probes on the surface can range from about 1,000 to 200,000 probe molecules per square micron. The probe density can be controlled by adjusting the density of the reactive groups on the surface of the pad for either the in situ synthesis or post-synthesis deposition methods. Other suitable means for synthesis of probe as are known in the art may be employed.

The oligonucleotide probes include, but are not limited to, the four natural deoxyribonucleotides; deoxythymidylic acid, deoxycytidylic acid, deoxyadenylic acid and deoxyguanylic acid. The probes can also be ribonucleotides, uridylic acid, cytidylic acid, adenylic acid, and guanylic acid. Modified nucleosides may also be incorporated into the oligonucleotide probes. These include but are not limited to; 2′-deoxy-5-methylcytidine, 2′-deoxy-5-fluorocytidine, 2′-deoxy-5-iodocytidine, 2′-deoxy-5-fluorouridine, 2′-deoxy-5-iodo-uridine, 2′-O-methyl-5-fluorouridine, 2′-deoxy-5-iodouridine, 2′-deoxy-5(1-propynyl)uridine, 2′-O-methyl-5(1-propynyl)uridine, 2-thiothymidine, 4-thiothymidine, 2′-deoxy-5(1-propynyl)cytidine, 2′-O-methyl-5(1-propynyl)cytidine, 2′-O-methyladenosine, 2′-deoxy-2,6-diaminopurine, 2′-O-methyl-2,6-diaminopurine, 2′-deoxy-7-deazadenosine, 2′-deoxy-6methyladenosine, 2′-deoxy-8-oxoadenosine, 2′-O-methylguanosine, 2′-deoxy-7-deazaguanosine, 2′-deoxy-8-oxoguanosine, 2′-deoxyinosine or the like.

The polynucleotide probes can vary in length from a range of about 5 to about 100 nucleotides, such as about 8 to about 80 nucleotides, such as about 10 to about 60 nucleotides, and such as about 15 to about 50 nucleotides. Longer polynucleotide probes are typically employed for applications where the sample contains a high sequence-complexity target mixture. Shorter polynucleotide probes are typically employed in applications where single nucleotide discrimination, such as mutation detection, is desired.

The target molecule could be a nucleic acid such as genomic DNA, genomic RNA, messenger RNA, ribosomal RNA or transfer RNA, an oligonucleotide or polynucleotide of DNA or RNA generated by enzymatic process such as PCR or reverse transcription, or any synthetic DNA, RNA, or any other desired nucleic acid or any combination thereof. The target molecule may be double stranded or single stranded. It is preferred that the target molecule be single stranded in order to increase the efficiency of its interaction with the probe sequences. The target molecule could contain nanomaterials such a carbon nanotube, wherein the nanomaterial such as the carbon nanotube could be functionalized at its ends to molecules containing nucleic acid.

The architecture of the array probes may be either generic or specific with regard to the complementary target sequences that it may hybridize with. For example, an array of all possible 7-mer probe sequences could be used to interrogate targets having any sequence. The advantage of such an array is that it is not application specific and therefore generic. Alternatively, the probe array may contain polynucleotide sequences that are complementary to a specific target sequence or set of target sequences and individual or multiple mutations thereof. Such an array is useful in the diagnosis of specific disorders, which are characterized by the presence of a particular nucleic acid sequence. For example, the target sequence may be that of a particular exogenous disease causing agent, e.g. human immunodeficiency virus, or alternatively the target sequence may be that portion of the human genome which is known to be mutated in instances of a particular disorder, e.g., sickle cell anemia or cystic fibrosis, or to a portion of a genome known to be associated with certain phenotypes, e.g., resistance to certain drugs, over-reactivity to certain drugs, or even susceptibility to side-effects of certain drugs.

In one embodiment of the present invention, polymers on a plurality of dies on a wafer substrate are functionalized on the electrodes as follows. First, a terminal end of a monomer, nucleotide, or linker molecule (i.e., a molecule which “links,” for example, a monomer or nucleotide to a substrate) is provided with at least one reactive functional group, which is protected with a protecting group removable by an electrochemically generated reagent. The protecting group(s) is exposed to reagents electrochemically generated at the electrode and removed from the monomer, nucleotide or linker molecule in a first selected region to expose a reactive functional group. The substrate is then contacted with the monomer or a pre-formed molecule (called the first molecule) such that the surface bonds with the exposed functional group(s) of the monomer or the pre-formed molecule. The first molecule may also bear at least one protected chemical functional group removable by an electrochemically generated reagent. The monomer or pre-formed molecule can then be deprotected in the same manner to yield a second reactive chemical functional group. A different monomer or pre-formed molecule (called the second molecule), which may also bear at least one protecting group removable by an electrochemically generated reagent, is subsequently brought in the vicinity of the substrate to bond with the second exposed functional group of the first molecule. Any unreacted functional group can optionally be capped at any point during the synthesis process. The deprotection and bonding steps can be repeated sequentially at the plurality of the predefined regions on the substrate until polymers or oligonucleotides of a desired sequence and length are obtained.

In another embodiment of the present invention, polymers on a plurality of dies on a wafer substrate are functionalized on the electrodes as follows. First, a substrate of a wafer having one or more molecules bearing at least one protected chemical functional group bonded on an array of electrodes on a plurality of dies is obtained. The array of electrodes is contacted with a buffering or scavenging solution. Following application of an electric potential to selected electrodes in the array of electrodes sufficient to generate electrochemical reagents capable of deprotecting the protected chemical functional groups, molecules on the array of electrodes are deprotected to expose reactive functional groups, thereby preparing them for bonding. A monomer solution or a pre-formed molecule (called the first molecule), such as proteins, nucleic acids, polysaccharides, and porphyrins, is then contacted with the substrate surface of the wafer and the monomers or pre-formed molecules are bonded in parallel with a plurality of deprotected chemical functional groups on a plurality of dies on the wafer. Another sufficient potential is subsequently applied to select electrodes in the array to deprotect at least one chemical functional group on the bonded molecule or another of the molecules bearing at least one protected chemical functional group on a plurality of dies on the wafer. A different monomer or pre-formed molecule (called the second molecule) having at least one protected chemical functional group is subsequently bonded to a deprotected chemical functional group of the bonded molecule or the other deprotected molecule located at a plurality of dies of the wafer. The selective deprotection and bonding steps can be repeated sequentially until polymers or oligonucleotides of a desired sequence and length are obtained. The selective deprotection step is repeated by applying another potential sufficient to effect deprotection of a chemical functional group on a bonded protected monomer or a bonded protected molecule. The subsequent bonding of an additional monomer or pre-formed molecule to the deprotected chemical functional group(s) until at least two separate polymers or oligonucleotides of desired length are formed on the substrate.

The embodiments of the invention can be used to carry out the electrochemical syntheses of polymers such as DNA and peptides according to any of a variety of approaches known to person skilled in the art. For example, any of a variety of reduction/oxidation (redox) reactions may be employed to electrochemically control the localization and pH of a solution on Si-based electrodes to enable the attachment and elongation of polymers. In such methods, the electrical current drives the oxidation of an appropriate molecule at the anode(s) and the reduction of another molecule at the cathode(s) to control the kinetics and stoichiometry of acid-catalyzed organic syntheses on a Si-based circuit Such methods can also be used to generate high pH (basic) solutions, and to drive any other electrochemical redox reactions known to one skilled in the art that may or may not result in pH changes (e.g., can also be used to generate reactive free radicals).

Another embodiment of the invention is electrochemical detection using the array chip. Typically these methods employ measurements of current flow across a DNA monolayer tethered to a circuit on a silicon substrate. Current flow properties proportionately change when the DNA monolayers are bound by an appropriate redox molecule-tagged test DNA or untagged DNA that is co-added with a redox-active molecule that specifically binds double stranded DNA. Enzyme amplification methods can also be incorporated into such assays in order to enhance the electrochemical signal generated by binding events. Note that these methods can also be adapted by one skilled in the art to measure the binding between other molecular species such as between two proteins or a protein and a small molecule.

The array chip could also be used for therapeutic materials development, i.e., for drug development and for biomaterial studies, as well as for biomedical research, analytical chemistry, high throughput compound screening, and bioprocess monitoring. An exemplary application includes applications in which various known ligands for particular receptors can be placed on the array chip and hybridization could be performed between the ligands and labeled receptors.

Yet another application of the array chip of an embodiment of this invention includes, for example, sequencing genomic DNA by the technique of sequencing by hybridization. Non-biological applications are also contemplated, and include the production of organic materials with varying levels of doping for use, for example, in semiconductor devices. Other examples of non-biological uses include anticorrosives, antifoulants, and paints.

It is specifically contemplated that the array chip and/or the methods of manufacturing the array chip of an embodiment of the invention could be used for developing new materials, particularly nanomaterials for many purposes including, but not limited to corrosion resistance, battery energy storage, electroplating, low voltage phosphorescence, bone graft compatibility, resisting fouling by marine organisms, superconductivity, epitaxial lattice matching, or chemical catalysis. Materials for these or other utilities may be formed proximate to one or a plurality of the electrodes in parallel on a plurality of dies of a silicon wafer, for example. Alternatively, materials may be formed by modifying the surface of one or a plurality of electrodes on a plurality of dies by generating reagents electrochemically.

It is further contemplated that an array chip of the embodiments of the invention could be used to develop screening methods for testing materials. That is, reagents electrochemically generated by an electrode on a die could be used to test the physical and chemical properties of materials proximate to the electrode. For example, the array chip could be used for testing corrosion resistance, electroplating efficiency, chemical kinetics, superconductivity, electro-chemiluminescence and catalyst lifetimes.

The advantageous characteristics of some of the embodiments of the invention are illustrated in the examples, which are intended to be merely exemplary of the invention.

The array chips of the embodiments of the invention are preferably silicon bio-chips built by using silicon process technology and SRAM like architecture with circuitries including electrode arrays, decoders, serial-peripheral interface, on chip amplification, for example.

The embodiments of this invention have several practical uses. For example, one embodiment of the invention allows molecules and nanomaterials detection/analysis based on the electrical readout of specific binding events (target to functionalized electrodes with probes) using CMOS-based devices. Another embodiment of the invention has potential applications for nanomaterials study (for example, in-situ analysis of DNA-mediated assembly of carbon nano-tubes on functionalized electrodes) to be used in electronic devices (CNT transistors and interconnects) as well as well as for detection of bio-species (DNA, protein, viruses etc.) for molecular diagnostics, homeland security, drug discovery and life science R&D work. Yet another embodiment of the invention could be to use Nanomaterials, such as carbon-nanotubes, in potential applications as interconnect materials. Carbon-nanotubes have lower resistivity than Cu and higher electromigration resistance (1000×higher than Cu). Yet another application could be to develop DNA functionalized electrodes with CMOS circuitry for immobilizing, detection, addressing, electrical readout and amplification of the signal can find potential application in silicon DNA chips. Silicon chips with DNA functionalized electrodes could find potential application to build nano-structures and in-situ assembly study of nanomaterials. Silicon DNA chips could also find potential application in medical diagnostics, homeland security devices, drug discovery and life science R&D work.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A device comprising a functionalized electrode having a probe molecule, wherein the device has an ability to electrically detect a molecular binding event between the probe molecule and a target molecule by a polarization change of the functionalized electrode.

2. The device of claim 1, further comprising an unfunctionalized electrode that does not have the probe molecule, wherein the device has an ability to electrically detect the molecular binding event between the probe molecule and the target molecule by a polarization change between the functionalized electrode and the unfunctionalized electrode.

3. The device of claim 1, further comprising a differential amplifier to amplify a current generated by the polarization change of the functionalized electrode.

4. The device of claim 2, further comprising a differential amplifier, wherein the differential amplifier is to amplify a current generated by the polarization change between the functionalized electrode and the unfunctionalized electrode.

5. The device of claim 1, further comprising a substrate comprising a wafer.

6. The device of claim 1, further comprising a switch and a capacitor, and wherein the polarization change modulates a gate of the field effect transistor.

7. The device of claim 1, wherein the probe molecule and the target molecule are label-free.

8. The device of claim 1, wherein the target molecule is a single-stranded DNA, RNA, protein or a nanomaterial functionalized with DNA.

9. The device of claim 1, wherein the probe molecule comprises a complementary molecular probe attached to the functionalized electrode.

10. The device of claim 1, wherein the device is a CMOS-based charge sensor and the device is not a current-voltage redox sensor.

11. A method of manufacturing a device comprising functionalizing a first electrode with a probe molecule to form a functionalized electrode and not functionalizing a second electrode to form an unfunctionalized electrode, wherein the device has an ability to electrically detect a molecular binding event between the probe molecule and a target molecule by a polarization change between the functionalized electrode and the unfunctionalized electrode.

12. The method of claim 11, further comprising fabricating a differential amplifier.

13. The method of claim 12, wherein the differential amplifier is to amplify a current generated by the polarization change between the functionalized electrode and the unfunctionalized electrode.

14. The method of claim 11, further comprising fabricating an interface logic.

15. The method of claim 11, wherein the device has the ability to electrically detect the molecular binding event without labeling the probe molecule or the target molecule.

16. A method of measuring a molecular binding event between a probe molecule and a target molecule, comprising obtaining a device comprising an unfunctionalized electrode and a functionalized electrode having the probe molecule, and detecting the molecular binding event between the probe molecule and the target molecule by a polarization change between the functionalized electrode and the unfunctionalized electrode.

17. The method of claim 16, wherein the device further comprises a differential amplifier, wherein the differential amplifier is to amplify a current generated by the polarization change between the functionalized electrode and the unfunctionalized electrode.

18. The method of claim 16, further modulating a gate of the field effect transistor by the polarization change.

19. The method of claim 16, wherein the target molecule is a single-stranded DNA, RNA, protein or a nanomaterial fuictionalized with DNA.

20. The method of claim 16, wherein the probe molecule comprises a complementary molecular probe attached to the functionalized electrode.

21. A method of manufacturing a circuit comprising attaching a first molecule capable of undergoing a hybridization event to a first end of a nanomaterial and hybridizing the first molecule to a second molecule located on a first pad of a die.

22. The method of claim 21, further comprising attaching a third molecule of a second of the nanomaterial and hybridizing the third molecule to a fourth molecule located on a second pad of the die.

23. The method of claim 22, wherein the nanomaterial forms an electrically conductive path between the first and second pads.

24. The method of claim 23, wherein the nanomaterial is a carbon nanotube, a nanowire or a nanopoarticle.

25. The method of claim 21, wherein the first pad comprises an electrode and the hybridization is coupled with generation of a catalytic current on the electrode.

26. A die comprising a first pad, a second pad and a nanomaterial connecting the first and second pads, wherein the nanomaterial is connected to the first and second pads by a hybridized molecule.

27. The die of claim 26, wherein the nanomaterial is a carbon nanotube, a nanowire or a nanoparticle.

28. An electrode having a three dimensional shape of a via.

29. The electrode of claim 28, wherein the via is about 1 to 10,000 micron in width and about 1 to 10 microns in deep.

30. The electrode of claim 28, wherein the via has a bottom wall and side walls.

31. A test device comprising (a) a first metal layer comprising a functionalized electrode comprising a probe molecule and (b) a second metal layer comprising a second electrode that can be resistively heated to cause a target molecule to de-hybridize, wherein the test device is to study de-hybridization of the target molecule.

32. The test device of claim 31, further comprising a third metal layer comprising a third electrode that can be resistively heated to cause the target molecule to de-hybridize.

33. The test device of claim 31, wherein the probe molecule is selected to such that the probe molecule can withstand a temperature of up to about 100° C.

34. The test device of claim 31, wherein the test device has an ability to electrically detect a molecular de-binding event between the probe molecule and the target molecule by a polarization change of the functionalized electrode

35. The test device of claim 31, wherein the first metal layer further comprises an unfunctionalized electrode that does not have the probe molecule, wherein the test device has an ability to electrically detect the molecular de-binding event between the probe molecule and the target molecule by a polarization change between the functionalized electrode and the unfunctionalized electrode.

36. The test device of claim 31, wherein the target molecule is a single-stranded DNA, RNA, protein or a nanomaterial functionalized with DNA and the test device is to study enzymatic or temperature-induced de-hybridization of the target molecule.

37. The test device of claim 1, wherein the functionalized electrode has a three dimensional shape of a via comprising a bottom wall and side walls.

38. The test device of claim 37, wherein the via is about 1 to 10,000 micron in width and about 1 to 10 microns in deep.

39. The test device of claim 2, wherein the functionalized electrode and the unfunctionalized electrode have a three dimensional shape of a via having a bottom and side walls.

40. The test device of claim 39, wherein the via is about 1 to 10,000 micron in width and about 1 to 10 microns in deep.

41. A device for trapping a target molecule comprising a first electrode, a second electrode and a third electrode, wherein the first, second and third electrodes are independently addressable electrodes, and wherein the second and third electrodes overlap the first electrode and contains a channel above the first electrode to permit the target molecule to be trapped into the channel.

42. The device of claim 41, wherein the target molecule is a charged target molecule and wherein the second and third electrodes have a voltage difference to produce an electric field between the second and third electrodes to ensure that the charged target molecule is attracted into the channel.

43. The device of claim 41, further comprising a probe molecule attached to the first electrode.

44. The device of claim 43, wherein the probe molecule is a cDNA probe or a polynucleotide probe or a nanomaterial functionalized with DNA.

45. The device of claim 41, further comprising a CMOS circuitry comprising a switching scheme for individually addressing the first, second and third electrodes.

46. The device of claim 41, further comprising a metal layer between the first electrode and the second electrode and another metal layer between the second electrode and the third electrode.

47. The device of claim 42, wherein the charged target molecule is a charged DNA, a charged nanomaterial, RNA, a protein or a nanomaterial modified with a charged molecule.

48. The device of claim 41, wherein one end of the channel that meets the first electrode is closed and the other end of the channel is open to ensure movement of a target molecule comprising a DNA through the open end of the channel.

49. The device of claim 41, further comprising a polymer brush attached to the first electrode.

50. The device of claim 41, wherein the second and third electrodes are ring shaped.

51. A method for manufacturing a device for trapping a target molecule, comprising forming a first electrode on a substrate, forming a second electrode on the first electrode, a forming third electrode on the second electrode, and forming a channel in the second and third electrodes, wherein the first, second and third electrodes are independently addressable electrodes and the channel above the first electrode is to permit the target molecule to be trapped into the channel.

52. The method of claim 51, wherein the channel ends at the top of the first electrode.

53. The method of claim 51, further comprising depositing a metal layer between any two of the first, second and third electrodes.

54. The method of claim 51, further comprising depositing a silicon-containing layer.

55. The method of claim 51, wherein the second and third electrodes are shaped like a ring.

56. The device of claim 8, wherein the nanomaterial is carbon, a nanotube, a nanowire or a nanoparticle.

57. The devise of claim 9, wherein the complementary molecular probe is DNA, RNA or an anti-body.

58. The method of claim 21, wherein the first molecule is a polynucleotide.

59. The method of claim 21, wherein the second molecule is a polynucleotide.

60. The method of claim 22, wherein the third molecule is a polynucleotide and the fourth molecule is a polynucleotide.
